(19) United States
(12) Patent Application Publication
(10) Pub. No.: US 2006/0270852 A1
(43) Pub. Date: Nov. 30, 2006

(54) PREPARATION OF BISQUINOLINE COMPOUNDS

(76) Inventors: Gyan Chand Yadav, Ghaziabad (IN); S. Srinivasan, Gurgaon (IN); Manjunath Govind Bhovi, North Kanara (IN); Riteshkumar Girishkumar Patel, Karamsad (IN)

Correspondence Address: Jayadeep R. Deshmukh, Esq.;Ranbaxy Inc., Suite 2100, 600 College Road East, Princeton, NJ, 08540, US

(21) Appl. No.: 11/420,400

(22) Filed: May 25, 2006

(30) Foreign Application Priority Data
May 30, 2005 (IN) 1384/DEL/2005

(51) Int. Cl. A61K 31/497; C07D 403/14

(52) U.S. Cl. 544/357

(57) ABSTRACT

The present invention provides a simple and cost-effective process for the preparation of a bisquinoline compound and its acid addition salts thereof.

### FIELD OF THE INVENTION

This invention relates to the preparation of a bisquinoline compounds having antimalarial activity.

### BACKGROUND OF THE INVENTION

1,3-Bis-[4-(7′-chloro-quinoline-4′)piperazine-1]propane of Formula I, commonly known as piperaquine is an antimalarial compound that belongs to the bisquinoline class of chemical compounds.

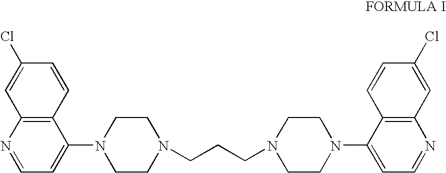

The bisquinoline antimalarial compound of Formula I was first synthesised in the 1960s (U.S. Pat. No. 3,173,918) and used extensively in China and Indochina as prophylaxis and treatment.

It is a highly lipid-soluble drug with a large volume of distribution at steady statelbioavailability, long elimination half-life and a clearance that is markedly higher in children than in adults. The tolerability, efficacy, pharmacokinetic profile and low cost of piperaquine make it a promising partner drug for use as part of artemisinin combination therapies. (Drugs, (2005), 65, 1, pp. 75-87).

A number of Chinese research groups documented that it was at least as effective as, and better tolerated than, chloroquine against falciparum and vivax malaria. With the development of piperaquine-resistant strains of Plasmodium falciparum and the emergence of the artemisinin derivatives, its use declined during the 1980s. (Drugs, (2005), 65, 1, pp. 75-87)

However, during the next decade, piperaquine was rediscovered by Chinese scientists as a molecule suitable for combination with an artemisinin derivative. The rationale for artemisinin combination therapy was to provide an inexpensive, short-course treatment regimen with a high cure rate and good tolerability that would reduce transmission and protect against the development of parasite resistance.

Piperaquine is available as a free base and also as its water-soluble tetra-phosphate salt, piperaquine phosphate of Formula II as shown below:

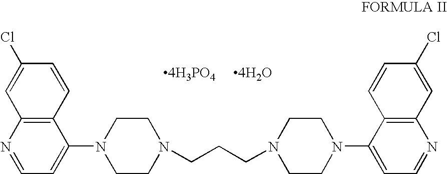

Piperaquine base is a pale white to yellow crystalline powder with a melting point of 212-213° C. [J. Chromatogr. B Analyt. Technol. Biomed. Life Sci. (2003), 791, 93-101] and UV absorption peaks at 225, 239 and 340 nm (“Meeting on Antimalarial Drug Development”; (2001) November 16-17; Shanghai, China: World Health Organization Regional Office for the Western Pacific, Manila: World Health Organization, (2002): 41-52). It is a basic compound (dissociation constant [pKa]=8.92) that is only sparingly soluble in water at neutral and alkaline pH, but has high lipid solubility (log₁₀ P=6.16).

Piperaquine phosphate is a white to pale yellow crystalline powder, soluble in water, slightly bitter, sensitive to light and has a melting point 246-252° C. (B. Med. Sci. Thesis, Crawley (WA): University of Western Australia, 2002). Although commercially available in China and listed in the Chinese Pharmacopoeia, piperaquine phosphate is not yet included in Western pharmacopoeias (Drugs, (2005), 65, 1, pp. 75-87).

Various processes for the preparation of piperaquine are known from the prior art. U.S. Pat. No. 3,173,918 (hereinafter referred to as “the '918 patent”) discloses the piperaquine molecule and its non-toxic acid addition salts. In this patent, Examples I, VIII and XVII describe the preparation of piperaquine. Condensation of 4,7-dichloroquinoline with 1,3-bis-1′-piperazinylpropane is performed in the presence of phenol in Example I of the '918 patent, and the resultant mixture is poured into an aqueous base solution to obtain piperaquine after recrystallization with dimethylformamide. Example VIII of the '918 patent discloses the condensation of 7-chloro-4-1′-piperazinyl-quinoline with 1,3-dibromopropane in the presence of triethylamine and methyl ethyl ketone to obtain piperaquine after recrystallization with ethanol. In the same patent, condensation of 1-(1-7′-chloro-4′-quinolyl-4-piperazinyl)-3-1′-piperazinylpropane with 4,7-dichloro quinoline is exemplified in Example XVII in the presence of phenol. After condensation, the mass obtained is poured into an aqueous basic solution and then piperaquine is obtained after column chromatographic purification. In this example, piperaquine is recrystallized with acetonitrile solvent. In Example XVII, 1-(1-7′-chloro-4′-quinolyl-4-piperazinyl)-3-1′-piperazinylpropane is prepared by the removal of the ethoxycarbonyl group from 1-(1-7′-chloro-4′-quinolyl-4-piperazinyl)-3-(1-ethoxycarbonyl-4-piperazinyl)propane, itself prepared by the reaction of 1-(7-chloroquinolyl)-4-(3-chloropropyl)piperazine on the hydrochloride of 1-ethoxycarbonylpiperazine.

7-chloro-4-(piperazin-1-yl)quinoline of Formula III as shown below, is one of the intermediates for the preparation of piperaquine.

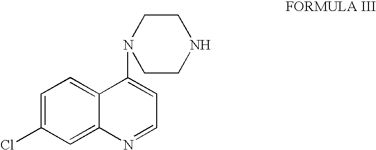

U.S. Pat. No. 3,331,843 (hereinafter referred to as “the '843 patent”) exemplifies a process of preparation of the intermediate of Formula III by the condensation of 4,7-dichloroquinoline with anhydrous piperazine in the presence of phenol. The condensation product is then sequentially extracted from its aqueous acidic solution by using solvents, for example, ether, benzene and the like, and the analytically pure intermediate is obtained by recrystallization from cyclohexane. In the '843 patent, conversion of the intermediate of Formula III to piperaquine is not described.

The preparation of intermediate is also disclosed in J. Med. Chem. (1998), 41, 4360-4364 and J. Med. Chem. (1971), 14(4), 283-286 but these references are silent about the conversion of the intermediate into piperaquine or its non-toxic acid addition salts.

All the above cited prior-art processes have certain disadvantages including, for example long synthetic approaches involving many steps, use of highly expensive solvents, low yield of the reaction products as well as intermediates, difficulties of scale-up of protection and deprotection during the synthesis, use of toxic materials and large quantity of solvents like phenol, dimethylformamide, methyl ethyl ketone and hence increases aqueous waste streams and the environment burden. Above all, the prior-art processes are costlier and complex too.

### SUMMARY OF THE INVENTION

It has now been discovered that simple, high yielding and cost effective processes for the preparation piperaquine are available.

While working on the above problem, it has been found that the intermediate of Formula III can directly be converted into piperaquine without the use of organic solvent during a condensation step. The present process is simple, cost-effective and the probability of the formation of impurities is very low. The piperaquine prepared by the process of the present invention can further be converted into its acid addition salts.

### DETAILED DESCRIPTION OF THE INVENTION

In one aspect, a process for preparation of piperaquine and acid addition salts thereof is provided, which comprises:
a) condensing 7-chloro-4-(piperazin-1-yl)quinoline compound of Formula III with 1,3-dibromopropane in presence of water, base and in the absence of an organic solvent to obtain piperaquine of Formula (I); and
b) optionally converting the product of Formula (I) into its acid addition salts.

7-chloro-4-(piperazin-1-yl) quinoline of Formula III can be prepared by known processes. The compound of Formula III can be prepared, for example by condensing 4,7-dichloroquinoline with anhydrous piperazine in presence of base and an organic solvent. The base can be, for example, potassium carbonate, and isopropyl alcohol, for example, can be used as an organic solvent. Any conventional base and organic solvent can be used during the condensation.

Piperaquine of Formula (I) can be prepared by condensing the compound of formula (III) with 1,3-dibromopropane in deionised water, base, in the absence of an organic solvent.

The base can be for example, any of hydroxides, carbonates, bicarbonates, sulphates, bisulphates, each of alkali metals or alkaline earth metals (for example, sodium carbonate, potassium carbonate, calcium carbonate, magnesium carbonate, sodium hydroxide, potassium hydroxide, calcium hydroxide, magnesium hydroxide, sodium sulphate, calcium sulphate, potassium sulphate, magnesium sulphate, sodium bicarbonate, potassium bicarbonate, calcium bicarbonate, magnesium bicarbonate or mixtures thereof). In particular the base can be sodium carbonate.

Crude piperaquine obtained after the condensation step can be further washed with deionised water till neutral pH is reached and then it can be refluxed with an organic solvent such as alkanols, ketones, esters, hydrocarbons, or chlorinated hydrocarbons, ethers or polar aprotic solvents and/or mixtures thereof. After refluxing, the product can be filtered and washed with organic solvent. In particular, the organic solvent can be denatured spirit. The product is dried to obtain pure piperaquine.

The piperaquine can be further converted to its acid addition salts by following known processes. The acid addition salts may be obtained by the action of an acid on the piperaquine compound in an appropriate solvent, for example, water and/or water-miscible solvents, alcohols, ethers, esters, ketones and/or mixtures thereof. Water is suitable as a solvent. The acid addition salts so obtained comprise hydrochloride and other hydrohalides, phosphates, nitrates, sulphates, acetates, propionates, succinates, benzoates, fumarates, maleates, theophylline-acetates, salicylates, phenolphathalinates, methylene-bis-β-hydroxynaphthoates (also known as embonates), resorcylates, gentisates and p-hydroxyisophthalates, and the like.

Piperaquine phosphate of Formula II prepared by the above process can be a purity of, for example, 99.68% or more.

Compounds analogous to piperaquine or acid addition salts thereof can also be prepared by following the same basic chemistry as mentioned above, e.g, chlorine substituent in 4,7-dichloroquinoline, compounds of Formula (I), (II) and (III) can suitably be substituted or replaced by other atoms or organic groups. Similarly, dihaloalkane or its aromatic or alicyclic analogues can be used in place of 1,3-dibromopropane. Piperazine may be substituted with other atoms or unsubstituted can also be used in the reaction.

The term ‘other atoms’ as used herein above includes hydrogen, bromine, fluorine, iodine, branched or unbranched alkyl or cycloalkyl group having 1-6 carbon atoms which are further substituted or unsubstitued, aryl group which may be substituted or unsubstituted, sulphate, phosphate or the like.

The organic groups can include, for example alcohol, ethers, ketones, aldehydes, esters, carboxylic acids, amides, nitriles or isonitriles or the like.

Dihaloalkane or its aromatic or alicyclic analogues compounds can include, for example branched, substituted with other atoms or unsubstituted hydrocarbons having 1-8 carbon atoms.

The following examples illustrate particular aspects. However, they do not limit the scope of the present invention. Variants of these examples would be evident to persons ordinarily skilled in the art.

### EXAMPLE 1

### Preparation of 7-chloro-4-(piperazin-1-yl) quinoline

A solution of 4,7-dichloroquinoline (59.4 g, 1 equivalent mole), piperazine (77.4 g, 3 equivalent mole)and potassium carbonate (41.4 g, 1 equivalent mole) in isopropyl alcohol (594 ml) was refluxed for 36 hours at 84-85° C. The mixture was cooled and then reheated to distill the solvent under reduced pressure. Water (1200 ml) was added into the reaction mixture, and the aqueous layer was extracted twice with dichloromethane (207 ml). The combined organic layer was concentrated and it was prolonged evacuated under low pressure. Hexane (240 ml) was added into the reaction mass and it was stirred for hour at room temperature, which afforded an off-white crystalline solid. The contents were filtered and washed with hexane (60 ml) and dried at 50-60° C. under vacuum for four hours to give 7-chloro-4-(piperazine-1-yl)quinoline (70.68 g), m.p. 113-115° C.
Yield: 1.19 w/w.
Purity (by HPLC): 96.64%.

### EXAMPLE 2

### Preparation of Piperaquine

A mixture of 7-chloro-4-(piperazine-1-yl) quinoline (40 g, 1 equivalent mole), 1,3-dibromopropane (16.15 g, 0.5 equivalent mole), sodium carbonate (20.4 g, 1.2 equivalent mole) in deionised water (400 ml) was heated under reflux for 15 hours at 100° C. The reaction mixture was cooled to room temperature and filtered. The product was washed with deionised water till neutral pH was achieved. The product was again heated in denatured spirit for two hours. Reaction mixture was cooled to room temperature and solid material was filtered and dried at 50-60° C. under vacuum for four to five hours to give 1,3-bis(1-7′-chloro-4-quinolyl-4-piperazinyl)propane (30 g).
Yield: 0.8 w/w.
Purity (by HPLC): 99.06%

### EXAMPLE 3

### Preparation of Piperaquine Phosphate

A suspension of 1,3-bis(1-7′-chloro-4-quinolyl-4-piperazinyl)propane (100 g, 1 equivalent mole) in water (1500 ml) was cooled up to 5-15° C. with stirring. A pre-prepared solution of ortho-phosphoric acid (85 ml, 4.0 equivalent) in water (500 ml) was drop wise added to the suspension during a period of 2 to 3 hours. The solution was allowed to stir for two hour at the same temperature. The solid product was filtered and washed with water (200 ml). The product was dried at 50-55° C. under high vacuum till water content reached 6-8% to obtain the title compound (170 g), m.p. 246-252° C.
Yield: 1.70 w/w.
Purity (by HPLC): 99.68%

We claim:

1. A process for preparation of piperaquine of Formula (I) and acid addition salts thereof, which comprises:
a) condensing 7-chloro-4-(piperazin-1-yl)quinoline compound of Formula III 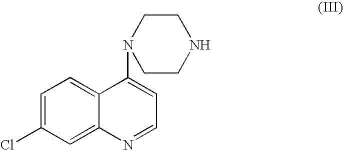
with 1,3-dibromopropane in the presence of water, base and in the absence of an organic solvent to obtain piperaquine of Formula (I); and 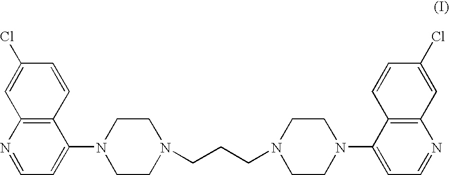
b) optionally converting the product of Formula (I) into its acid addition salts.

2. The process of claim 1 wherein the base is selected from sodium carbonate, potassium carbonate, calcium carbonate, magnesium carbonate, sodium hydroxide, potassium hydroxide, calcium hydroxide, magnesium hydroxide, sodium sulphate, calcium sulphate, potassium sulphate, magnesium sulphate, sodium bicarbonate, potassium bicarbonate, calcium bicarbonate, magnesium bicarbonate or mixtures thereof.

3. The process of claim 2, wherein the base is sodium carbonate.

4. The process of claim 1, wherein acid addition salts are selected from hydrochlorides and other hydrohalides, phosphates, nitrates, sulphates, acetates, propionates, succinates, benzoates, fumarates, maleates, theophylline-acetates, salicylates, phenolphathalinates, methylene-bis-β-hydroxynaphthoates (also known as embonates), resorcylates, gentisates and p-hydroxyisophthalates.

5. The process of claim 4, wherein acid addition salts are phosphates.

6. Piperaquine phosphate of Formula (II) prepared by the process of claim 1.

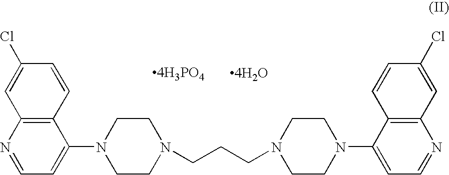

7. Piperaquine phosphate of Formula II, prepared by the process of claim 1, having purity of above 99%.
